(19) United States
(12) Patent Application Publication
(10) Pub. No.: US 2009/0281437 A1
(43) Pub. Date: Nov. 12, 2009

(54) Application of Nanotechnology for Blood Flow Meters

(76) Inventor: Alexander Grinberg, San Francisco, CA (US)

Correspondence Address: Dr. Alexander Grinberg, 600 15th Avenue, San Francisco, CA, 94118, US

(21) Appl. No.: 12/464,644

(22) Filed: May 12, 2009

Related U.S. Application Data
(60) Provisional application No. 61/052,304, filed on May 12, 2008.

(51) Int. Cl. A61B 5/02

(52) U.S. Cl. 600/504; 977/904

(57) ABSTRACT

Conventional blood flow meter can be combined with photonic crystals to measure temperature change for heat washin/washout. These photonic crystals are impregnated into conventional devices as a thermometer. It makes the entire device more compact and easier for patients.

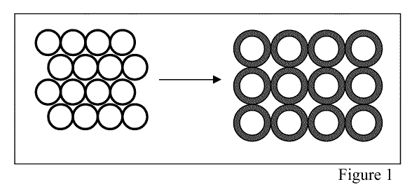

### BRIEF DESCRIPTION OF THE DRAWING

Transformation of colloidal crystals into new conformation due to response of sensitive surface layer of particles makes the average distance between particles higher and another lattice in photonic crystal. The color of the photonic crystal is also changed.

### DETAILED DESCRIPTION OF THE INVENTION

Idea is based on use of photonic crystals for detection of temperature (or mechanical) change. Currently a blood flow meter is used to measure heat washin/heat washout. I propose to use colloidal crystals (known as photonic crystals) for fast optical monitoring of temperature change.

Photonic crystals have a characteristic color depending on a so called “bandgap” relevant to size and interlayer distance between colloidal particles in the crystals. Color change response could be seen upon application of voltage or mechanical load.

Here, I propose to use particles with sensitive layer deposited on particle surface. It will be temperature sensitive polymer, for instance Poly(N-isopropyl acrylamide) (PNIPAM). Practically, it might be grafted chains to particle surface. PNIPAM or its analogues change their conformation while temperature is changing. Temperature of transformation could be tuned within range 30-40° C. to optimize the functionality of devices. Changing in average distance between particles causes color change of photonic crystals and can be easily seen. Temperature changes can be measured with expected precision of 0.05° C.

What is claimed is:

1. An apparatus where a conventional blood flow meter is combined with photonic crystals sensitive to temperature change

2. The apparatus of claim 1 where the photonic crystal is made temperature sensitive by surface modification of colloidal particles used for photonic crystal formation with polymers able to change conformation upon temperature change.

3. The apparatus of claim 1 where the temperature sensitive polymer may be grafted to the particle surface by any means.

4. The apparatus of claim 1 where tuning for increased sensitivity to temperature is done by choice of polymer and/or use of a co-polymer

5. The apparatus of claim 1 where temperature change is recorded by optical means via alternation of the average distance between particles in the photonic crystal
